(19) United States
(12) Patent Application Publication
(10) Pub. No.: US 2009/0092691 A1
(43) Pub. Date: Apr. 9, 2009

(54) Formulation Based on Marigold, Aloe, and Centellae

(76) Inventor: Lucia Antonia Spannagel, Cordoba (AR)

Correspondence Address: BACHMAN & LAPOINTE, P.C., 900 CHAPEL STREET, SUITE 1201, NEW HAVEN, CT, 06510, US

(21) Appl. No.: 12/114,997

(22) Filed: May 5, 2008

(30) Foreign Application Priority Data
May 3, 2007 (AR) P-07-01-01909

(51) Int. Cl. A61K 36/886

(52) U.S. Cl. 424/744

(57) ABSTRACT

The formulation includes between 1 and 98 parts of glycolic extract of marigold officinalis, between 1 and 98 parts of glycolic extract of aloe vera and between 1 and 98 parts of glycolic extract of centellae asiaticae. The formulation conforms an emulsion from the use of between 4 and 8% of glycolic extract of marigold officinalis, between 4 and 8% of glycolic extract of aloe vera, and between 5.5 and 9% of glycolic extract of centellae asiaticae incorporated into a base cream non ionic that includes an oily phase where it is included between 6.5 and 12% of selfemulsive wax non ionic; between 3.2 and 5.8% of estearic acid of triple pressure; between 4 and 6.4% of solid Vaseline (petroleum jelly) and between 7.6 and 9.7% of liquid Vaseline (petroleum jelly). The watery phase includes between 7 and 10.4% of glycerine; between 0.1 and 0.8% of imidozolindil urea; between a 1.6 and 3.3% of preserving universal; between 0.7 and 1.5% of polyethilenglycol 400; between 0.35 and 0.78% of Twen 80. Finally the emulsion includes the addition of demineralized water up to completing 100 g. of product.

### BACKGROUND

(1) Field of the Invention

The present invention consists of a formulation based on marigold, aloe and centellae.

In order to make the present invention understandable so that it can be easily taken to practice, it will be given in the paragraphs that follow a precise description of a favorite form of accomplishment, and which formulation is exemplified incorporated into a basic form by a watery phase and an oily phase by purely demonstrative but not limitative character of the invention, which components will be able to combine with such diverse compounds as those who are exemplified in the report without it implying separating of the principles of the invention established in the present documentation.

(2) Prior Art

Innumerable compounds exist in the prior art with anti-inflammatory, antiseptic and healing properties. Also in the prior art the use of the compounds of the emulsion are well known. It is possible to say in general that such properties have been investigated necessarily from the times of our first ancestors in order to solve the continuous problems that were presented.

From observation, men have been progressing in the discovery of the properties of the beings that surround us and from the ingenuity such properties have sought to take advantage in their own benefit.

The plants and animals, from the need of food, have been the objects of the first observations, and later, those who have allowed to relate the reactions of the animals exposed to certain types of plants.

This is how one could determine the benign or harmful factors present in the plants.

The first ones were looked at when the animal needed to restore its health. The second ones were looked at by some species as defense from predators or avoided by them. Such there is at the time the origin of the medicines and poisons.

Throughout history, numerous species of plants have been studied. Still not all of them have been studied, partly because many remain ignored and partly because many others have disappeared already, and innumerable medicines have been achieved derived from its leaves, stems, flowers and roots.

Due to the fact that many of such species are known from long time ago, there is an extensive bibliography related to the studies, tests, results and analysis as turns out to be the case of the elements that are used in the formulation for which protection is solicited.

The marigold is an annual plant and is widely distributed all over the world. Its medicinal properties and colorings are well known from old times, especially for the Arabians and Indians and then for the Greeks.

Notes exist on the applications recommended by the Abbess Hildegarda of Bingen (1099-1179) in the treatment of the impetigo and dermal stains. It was considered of high therapeutic value by Albertus Magnus (1193-1280), being incorporated in almost all the herbal texts known about that time.

Closer in history, it can be seen that the father Sebastian Kneipp (1821-1897) was recommending the marigold for the treatment of sores, varicose veins and dermatological spots diseases. Diverse salves prepared on the basis of marigold have been applied like anti-inflammatory and antiseptic in the American Civil War as well as in the First World War.

Aloe is another of the principal ingredients of the formulation for which protection is being solicited. Aloe is a plant of the family of the liliaceous that also was recognized by its antiseptic and healing power from long data. There are known approximately 700 varieties of this plant that nowadays we use in the elaboration of so diverse products as food, paintings and cosmetics. Among the oldest quotations that can be found, the reference to the Bible is mandatory where it is reported how, after bringing down the cross the body of our Lord, was anointed with a salve prepared with myrrh and aloe.

Also, it appears in Dioscorides's herbalist's of the 1st century B.C. that Alexander the Great conquered the island Socotorra, South of Arabia, because there was finding of a great quantity of aloe, which would serve for the healing of wounds and diseases of his soldiers during the conquest. It is known that the Chinese were the first ones to use aloe, and in ancient Egypt also it was of frequent use.

For Ricardo Gampel, immune-pharmacologist and specialist in Natural Therapies of the University of Buenos Aires, the utilization of aloe goes together with the development of the humanity from its origins since it is documented among the Chinese, Indians, Sumerians, Assyrians, Babylonians, Egyptians and Hebrews. The most common uses were given in the treatment of wounds, injuries, burns, infections of the skin, indigestion and gases.

In the already mentioned herbalist, Dioscorides does a wide description of the plant for its medicinal and cosmetic properties. Introduced in the current Malaga for the Arabians, big extensions were cultivated until the modern pharmacopoeia relegated the aloe together with many other medicinal plants.

The rediscovery of this plant took place during the Second World War, when it was used in the treatment of the radioactive burns of the people from Hiroshima and Nagasaki.

The references indicate that the injuries recovered more quickly and in many cases without leaving signs or scars. More than 60% of the solids of the total of the aloe are polysaccharides mucilaginous ligatures to sugars as glucose, manose, ramnose, xilosa, arabinosa, galactosa and acids urónicos. The slime is composed of different neutral, acid polysaccharides and acetilados such as mananos, glucomananos, galactomananos, etc., which are in charge of the great capacity that has the plant to retain water, as well as the aloerido that is a polysaccharide of high molecular weight.

The remaining solid ones are organic salts and acids as the glutámico, málico, salicílico, citric, lactato magnesium, oxalato calcic, etc.; such enzymes as the celulasa, carboxipeptidasa, bradikininasa, catalasa, amylase, oxidasa and tirosinasa; sapogenics; tannins; esteroles; triglicerids; amino acids as the lisina, histidina, glutamina, arginina, aspartic acid, asparagina, treonina, serina, glutamic acid, glycine, alanina, valina, metionina, isoleucine, leucine, tirosine, fenilalanine and triptofano; RNA and traces of alkaloids; betacarotens; vitamins B1, B2, B3, B6, C, And; colina; folic acid and minerals like aluminium, boron, barium, calcium, chrome, copper, iron, potassium, magnesium, sodium, phosphorus, strontium and silicon.

It can be said that aloe is indicated for internal and external use in dermatological affections; exanthematic infections as measles, chicken pox, rubella and herpes; affections of mucous gastric and intestinal; gastrointestinal infections and inflammatory intestinal diseases as crohn, ulcerous colitis and irritable colon and of the buccal mucous aphthas, gingivitis, periodontitis, candidiasis mouth and esofagica), conditions(states) of immunosuppretion, inflammatory and autoimmune processes type arthritis, tumour processes, prevention of conditions(states) of immunosuppretion and infectious processes, hyperglycaemia and hyperlipidaemia.

Adapted for its use in processes of dermal cicatrization, the centellae regulates quantitatively and qualitatively the new conjunctive tissue allowing to obtain a soft, flexible tissue and not hypertrophic one. As an extract, the centellae regulates the metabolism of the conjunctive tissue stimulating the synthesis of collagen and of the glycosaminoglycans. It acts in the fibroblast of the conjunctive tissue intervening in the enzymatical activity that controls the fixation of prolina and alanina to the ARN matricials that will take part in the biosynthesis of collagen forming collagen I and collagen III as well as the normal skin and vasculars walls. It regulates the proliferation of the fibroblasts leading them to obtain, in quantity and quality of the required collagen, avoiding by this way the formation of keloids and orientates them towards the preferential synthesis of collagen. Is through the fibroblasts that controls the production of the collagenous fibers, when the process of cellular regeneration is disturbed or desorganized.

The centellae was known from ancient times and its leaves were used in Madagascar and India for the treatment of wounds, sores, ulcers and leprosy in the form of fresh juice of the plant.

In its composition the content emphasizes in saponins triterpenics, sugars, essential oils and tanins.

It possesses a great healing power on the basis of the stimulation of the cellular mitosis favoring the biosynthesis of the collagen to level the conjunctive tissues. Among the elements that can intervene in obtaining the results, one can find the aminados and abundant and varied mineral salts. The centellae is a very resistant climbing plant that grows in the tropics. Also known as “herb of the tiger” because hunters were following the track of these beasts and observing these plants of centellae, since the wounded animals had the habit of rolling about in the shrubs to recover.

Doing a search to determine the documents that could be considered to be precedents of the proposed invention, there have been located the following patents: CN1176134; CN1256128; CN1288741; CN1302654; CN1302661; CN1313106; CN1359701; CN1366992; CN1404858; CN1559580; CN1679643; DE10242138; DE19844255; JP2117618; JP5112458; JP57145806; RU2128500; US2005025723; U.S. Pat. No. 4,725,438; WO02069884 and WO2004039390, all which relate to compositions that include aloe or some derivative of aloe; however, none of the remaining components are revealed in this file.

Likewise there has been located the following U.S. Pat. Nos. 5,266,330; 5,843,467 and 5,997,876 in which the utilization of marigold is revealed, but as in case of the documents referring to the aloe, the same one is in use without the addition of any of the remaining components of the invention that is being revealed.

Finally there also has been located the documents: RU2138277; and RU2149009, that claim products and procedures where the above mentioned products have been elaborated on the basis of plants and herbs that do not guard relation with the developing in the present documentation.

### SUMMARY OF THE INVENTION

Basically the present invention consists of a formulation based on marigold, aloe and centellae obtained from glycolic extracts of flowers of marigold, air parts of centellae and gel of aloe that can be used as powder or talc, can join to a base and can include other components.

In a preferred form, the formulation that is revealed is used as an emulsion incorporating it into a base that presents an oily phase and a watery phase.

### DETAILED DESCRIPTION OF THE PREFERRED EMBODIMENT(S)

Once established the diverse components that explain the nature of the invention, it has to be complemented immediately afterwards with the functional and operative relation of the same as well as the result that they provide. In order to possess a product for the cure of diverse affections of the skin that favors the granulation and the later cicatrization, turning out to be a natural, non aggressive and humid product that proceeds to accomplishes a formulation based on marigold, aloe and centellae.

The first aspect that must be highlighted is that there is not known a formulation in which these three components appear together. From the research carried out and according to the documents founded as well as the consulted bibliography, it arises that each of the components has been studied for a long time across history and for such motive their properties have been taken advantage of under diverse preparations. Nevertheless the inventor is the one that, from her investigation and experimentation, has brought them together for the first time in the formulation that is being revealed.

In the instant document, there is specified a formula in which three components are incorporated to a base cream, and in the same one there are given the proportions for them. Nevertheless, according to the experience of the authoress, the three mentioned components can be incorporated in any proportion since the major or minor quantity of one of them with respect to the other two, it allows to obtain, not only different properties but also different physical characteristics with what the resultant formula of the different combinations allows to obtain a compound that will turn out to be more adapted for the treatment of a certain ailment, or it will give a consistency that will favor its use as lotion.

Among the characteristics of the product that is revealed it has the capacity of acting from the internal layers towards the exterior, provoking the drainage of the wound, for what if there was infection this one will not stay inside while the superficial layers are healing. The formulation includes then between 1 and 98 parts of a glycolic extract of marigold to which are added between 1 and 98 parts of a glycolic extract of aloe and between 1 and 98 parts of a glycolic extract of centellae.

A variant of the above mentioned formulation includes that, per every part of glycolic extract of marigold are added between 1 and 2 parts of a glycolic extract of aloe and between 1.3 and 2.4 parts of a glycolic extract of centellae.

The formulation can be used as a powder or talc, or can be incorporated into a base for its utilization as a paste, a solution, a lotion, a gel, a cream or an ointment.

The proposed formulation may be combined with other elements and chemical products such as excipients, antibiotics, vitamins and corticoids for example.

The preferred way of using the formulation that is being revealed consists of making an emulsion incorporating into a non-ionic base cream that has an oily phase and a watery phase.

In this form of emulsion is conformed from the incorporation of between 4 and 8% of glycolic extract of marigold, between 4 and 8% of glycolic extract of aloe and between 5.5 and 9% of glycolic extract from centellae to a non-ionic base cream in which the oily phase includes between 6.5 and 12% of selfemulsive wax non ionic; between 3.2 and 5.8% of estearic acid of triple pressure; between 4 and 6.4% of solid Vaseline (petroleum jelly) and between 7.6 and 9.7% of liquid Vaseline (petroleum jelly).

The watery phase of the above mentioned emulsion includes between 7 and 10.4% of glycerine; between 0.1 and 0.8% of imidozolindil urea; between a 1.6 and 3.3% of preserving universal; between 0.7 and 1.5% of polyethilenglycol 400; between 0.35 and 0.78% of Twen 80 and demineralized water up to completing the total.

In the present documentation one refers to marigold, to aloe and to centellae, it must be understood that such a reference is done bearing in mind that they are respectively the marigold officinalis, the aloe vera and the centellae asiaticae.

Also it has to be understood that in the proposed formula the percentage of each one of the active principles, must be understood like recounted to a relation volume/weight.

Finally, it corresponds to demonstrate that the glycolic extract of marigold is a transparent liquid of golden and soluble tone in water that has been obtained from the flowers of the plant.

In relation to the glycolic extract of the aloe, it consists of a transparent liquid of beige-golden soluble color of water and obtained from the gel of the plant.

Preferably, the above mentioned gel has been obtained by a procedure of stabilization that it turns out to be acquainted in the previous art.

In relation with the glycolic extract of centellae, it consists of a transparent liquid of golden tone that turns out to be soluble in water to a temperature of 25° C. (77 degrees F.) and is obtained of the air parts of the plant.

The glycolic extracts of marigold, aloe and centellae used in the formulation that is revealed, have a density of between 1.0 and 1.1 and 1.0 and 1.2 grams/milliliter and the pH of the same ones is considered between 4 and 7.

This way it is been outlined one of the constructive possibilities that lead to concrete the invention and also the way it works, and implying its specific applications.

1. A formulation based on marigold, aloe and centallae which includes between 1 and 98 parts of a glycolic extract of marigold officinalis, between 1 and 98 parts of a glycolic extract of aloe vera and between 1 and 98 parts of a glycolic extract of centellae asiaticae.

2. The formulation according to claim 1, wherein for every part of glycolic extract of marigold officinalis, there is added between 1 and 2 parts of extract of aloe vera and between 1.3 and 2.4 parts of glycolic extract of centellae asiaticae.

3. The formulation according to claim 1, wherein said formulation comprises an emulsion conformed by incorporation of between 4 and 8% of glycolic extract of marigold officinalis, between 4 and 8% of glycolic extract of aloe vera, and between 5.5 and 9% of glycolic extract of centellae asiaticae to a base cream non ionic that includes an oily phase to which has been added between 6.5 and 12% of selfemulsive wax non ionic; between 3.2 and 5.8% of estearic acid of triple pressure; between 4 and 6.4% of solid petroleum jelly and between 7.6 and 9.7% of liquid petroleum jelly and also including a watery phase in which are incorporated between 7 and 10.4% of glycerine; between 0.1 and 0.8% of imidozolindil urea; between 1.6 and 3.3% of preserving universal; between 0.7 and 1.5% of polyethilenglycol 400; between 0.35 and 0.78% of Twen 80 and demineralized water up to completing 100 g. of product; and which products that are included in the formulation are recounted to a percentage measured in relation to volume/weight.

4. The formulation according to claim 1, wherein the glycolic extract of marigold, the glycolic extract of aloe vera, and the glycolic extract of centellae are obtained respectively from flowers of the marigold, from a gel of the aloe, and from air parts of the centellae.

5. The formulation according to claim 1, wherein the glycolic extracts of marigold, aloe and centellae have a density that is between 1.0-1.2 grams/milliliter.

6. The formulation according to claim 1, wherein the pH of the glycolic extracts of marigold, aloe and centellae is between 4 and 7.

7. The formulation according to claim 1, wherein the gel of the aloe is obtained by estabilization.

8. The formulation according to claim 1, wherein the formulation is used as a powder or talc.

9. The formulation according to claim 1, wherein the formulation can be incorporated to a base for its utilization as a paste, a solution, a lotion, a cream, a gel or an ointment

10. The formulation according to claim 1, wherein the formulation can be combined with at least one additional constituent selected from the group consisting of excipients, antibiotics, corticoids, vitamins and a chemical product.
